(19) United States
(12) Patent Application Publication
(10) Pub. No.: US 2025/0340952 A1
(43) Pub. Date: Nov. 6, 2025

(54) MOLECULAR MARKER RELATED TO DIFFERENTIAL EXPRESSION OF PREGNANCY STAGE OF SHEEP AND APPLICATION THEREOF

(71) Applicant: Zhejiang University, Hangzhou (CN)

(72) Inventors: Zhengguang WANG, Hangzhou (CN); Shimin Qiu, Hangzhou (CN); Fengqin PAN, Hangzhou (CN); Hongqing WU, Hangzhou (CN); Zhonghua FEI, Hangzhou (CN); Yefeng WU, Hangzhou (CN); Hongwei CAO, Hangzhou (CN); Xi CHEN, Hangzhou (CN); Xin XIANG, Hangzhou (CN); Yanshe XIE, Hangzhou (CN)

(73) Assignee: Zhejiang University, Hangzhou (CN)

(21) Appl. No.: 19/093,297

(22) Filed: Mar. 28, 2025

(30) Foreign Application Priority Data
May 6, 2024 (CN) 202410547122.6

(51) Int. Cl. C12Q 1/6888; C12Q 1/26; C12Q 1/6809; C12Q 1/6851; G01N 33/50; G16B 25/10

(52) U.S. Cl. CPC C12Q 1/6888; C12Q 1/26; C12Q 1/6809; C12Q 1/6851; G01N 33/5023; G16B 25/10; C12Q 2600/124; C12Q 2600/178; G01N 2333/90241

(57) ABSTRACT

A molecular marker related to a differential expression of a pregnancy stage of sheep and application thereof is provided, the molecular marker is oar-miR-29a miRNA target gene, and a sequence is as follows: 5′-UAGCACCAUUUGAAAUCAGUGUU-3′ shown in SEQ ID NO: 12; the oar-miR-29a miRNA related to the pregnancy stage of sheep was screened and identified, through further experimental verification, the relationship between oar-miR-29a and CDC42 was found, which provided important clues for understanding the mechanism of miRNA regulatory network in the process of sheep pregnancy, it can be applied to regulate the uterine receptivity of sheep to improve the reproductive efficiency, and it can also be used as a molecular marker to optimize the breeding direction, cultivate new varieties or to make a 50K chip dedicated to Hu sheep.

### CROSS REFERENCE TO THE RELATED APPLICATIONS

This application is based upon and claims priority to Chinese Patent Application No. 202410547122.6, filed on May 6, 2024, the entire contents of which are incorporated herein by reference.

### SEQUENCE LISTING

The instant application contains a Sequence Listing which has been submitted in SML format via EFS-Web and is hereby incorporated by reference in its entirety. Said XML copy is named GBZYGJ303_Sequence Listing.xml, created on Feb. 14, 2025, and is 13,360 bytes in size.

### TECHNICAL FIELD

The present invention relates to agricultural genetic engineering, particularly to a molecular marker related to differential expression of pregnancy stage of sheep and application thereof.

### BACKGROUND

The improvement of the breeding efficiency of sheep is of great significance to animal husbandry. Uterine receptivity is the crucial part of sheep pregnancy, and microRNA (miRNA) plays an important regulatory role as a class of important regulators in the process of pregnancy. Therefore, it is of great application value to study the regulation of the differential expression of miRNA and its target genes in sheep during pregnancy.

It is necessary to screen a kind of miRNA related to pregnancy stage by bioinformatics analysis of the differential expression of miRNA in sheep uterus, which provides important clues for understanding the mechanism of regulatory network of miRNA in sheep pregnancy, and provides new methods and approaches for improving sheep reproductive efficiency. The present invention solves such a problem.

### SUMMARY

In order to solve the deficiencies of the existing technology, an objective of the present invention is to provide a molecular marker related to a differential expression of a pregnancy stage of sheep and the application thereof, the present invention screens and identifies an oar-miR-29a miRNA related to the pregnancy stage of sheep, discovers the relationship between oar-miR-29a and cell division cycle 42 (CDC42) through further experimental verification, and provides an important clue for understanding the mechanism of the regulatory network of miRNA during the pregnancy stage of sheep, which can be used for regulating the uterine receptivity of sheep to improve the breeding efficiency.

In order to achieve the above objectives, the present invention adopts the following technical scheme:
a molecular marker related to differential expression of a pregnancy stage of sheep, the molecular marker is oar-miR-29a miRNA target gene, and a sequence is as follows: 5′-UAGCACCAUUUGAAAUCAGUGUU-3′ shown in SEQ ID NO: 12.

The aforementioned molecular marker related to differential expression of the pregnancy stage of sheep, oar-miR-29a targets CDC42, and oar-miR-29a mimics inhibits CDC42 expression.

A method for screening the aforementioned molecular marker, the method includes the following steps:
step 1, analyzing the differential expression of miRNA in a plasma of pregnant sheep and non-pregnant sheep by a bioinformatics method:
comparing a composition of miRNA in the plasma of pregnant sheep and non-pregnant sheep in a public database, the results show that the composition of miRNA in the plasma of pregnant sheep is specific, and a group of miRNA with high differential expression abundance in the pregnancy stage are screened, wherein the group of miRNA is oar-let-7b, oar-miR-19b and oar-miR-29a, respectively;
step 2, screening out miRNA related to the pregnancy stage of sheep and its target genes:
using an online tool (bioinfo5.ugr.es/srnatoolbox) to predict the target gene of a target miRNA, and performing Kyoto Encyclopedia of Genes and Genomes (KEGG) analysis and Gene Ontology (GO) analysis on the target gene of the target miRNA to predict its correlation with embryo implantation, it can be seen that oar-miR-29a has a higher correlation with embryo implantation, constructing a protein-protein interaction (PPI) network for its target gene, and obtaining the following four genes located at key nodes: Albumin (ALB), Cluster of differentiation (CD) 80, CDC42, and signal transducer and activator of transcription (STAT); selecting the CDC42 with higher correlation with embryo implantation as a final target gene;
step 3, verifying miRNA and its target genes by experiments:
verifying the target gene CDC42 by fluorescence quantitative polymerase chain reaction (PCR) in Hu sheep endometrial epithelial cells transfected with oar-miR-29a mimics and inhibitor; PCR oar-miR-29a primer sequences include: specific primer: CTAGCACCATCTGAAATCGGTTA shown in SEQ ID NO: 1; U6 forward primer GGAACGATACAGAGAAGATTAGC shown in SEQ ID NO: 2; U6 reverse primer TGGAACGCTTCACGAATTTGCG shown in SEQ ID NO: 3; CDC42 forward primer GACCGCTGAGCTATCCAC shown in SEQ ID NO: 4; CDC42 reverse primer CTTGACAGCCTTCAGGTCAC shown in SEQ ID NO: 5;
after Hu sheep endometrial epithelial cells are transfected with oar-miR-29a mimics and inhibitors, the relative expression of oar-miR-29a increased and decreased, respectively, while the relative expression of CDC42 is trended in opposite to the relative expression of oar-miR-29a.

A method for verifying a targeting relationship between the aforementioned molecular marker and CDC42, including the following steps:
step 1, obtaining a binding sequence of oar-miR-29a and 3′ UTR of CDC42 from a database, constructing a CDC42 wild type (WT) plasmid vector, that is, CDC42 WT-oar-miR-29a, and constructing a CDC42 mutant type plasmid vector, that is, CDC42 MUT-oar-miR-29a; oar-miR-29a mimics sequence:
UAGCACCAUCUGAAAUCGGUUCCGAUUUCAGAUGGUGCUAUU shown in SEQ ID NO: 6;
oar-miR-29a inhibitor sequence: AACCGAUUUCAGAUGGUGCUA shown in SEQ ID NO: 7;
mimics negative control (NC) sequence: UUCUCCGAACGUGUCACGUTT ACGUGACACGUUCGGAGAATT shown in SEQ ID NO: 8;
inhibitor NC sequence: CAGUACUUUUGUGUAGUACAA shown in SEQ ID NO: 9; CDC42 WT-oar-miR-29a sequence:
CCTAGATCTAGTTTAGAGAACATGTTCCCCACCTGGTGCTCTTAGGAAGGAGT ATAGTAAACGCCTCAT shown in SEQ ID NO: 10;
CDC42 MUT-oar-miR-29a sequence:
CCTAGATCTAGTTTAGAGAACATGTTCCCGTCCACCACGACTTAGGAAGGAG TATAGTAAACGCCTCAT shown in SEQ ID NO: 11;
step 2, co-transfecting oar-miR-29a mimics and mimics NC respectively with CDC42 wild type/mutant plasmids into human embryonic kidney (HEK293T) cells; detecting a fluorescence activity of the transfected of the HEK293T cells by a dual-luciferase reporter gene assay system after transfection;
the fluorescence activity of the co-transfection group of CDC42 wild type plasmid and oar-miR-29a mimics is decreased.

An application for the aforementioned molecular markers, the application for the aforementioned molecular markers is applied to determine a direction of sheep breeding.

The application for the aforementioned molecular markers, the application for the aforementioned molecular markers is applied to optimize genetic characteristics of Hu sheep and to breed new lines.

The application for the aforementioned molecular markers, the application for the aforementioned molecular markers is applied to a 50K chip dedicated to Hu sheep, and a content of the 50K chip is a candidate gene, wherein the candidate gene takes the molecular marker as a basis and combines with other economic traits of Hu sheep.

The present invention is beneficial as follows:
the present invention provides a reliable method for predicting and verifying differentially expressed miRNAs and their target genes in sheep during pregnancy;
in the present invention, through the integration of bioinformatics analysis and experimental verification, the miRNA and its target gene oar-miR-29a related to sheep pregnancy can be accurately identified, the present invention proves that oar-miR-29a targets CDC42, which provides important clues for further understanding of sheep pregnancy regulatory network, and provides new methods and approaches for improving sheep breeding efficiency, and it can be applied to sheep breeding or used as a molecular marker to make a 50K chip dedicated to Hu sheep.

### Technical Term:

miRNA mimics mimic the endogenous miRNAs of organisms, they are synthesized by chemical synthesis methods and can enhance the function of endogenous miRNAs.

miRNA inhibitor is a chemically modified inhibitor that specifically target specific target miRNAs in cells.

### BRIEF DESCRIPTION OF THE DRAWINGS

FIG. 1 is a statistical graph of GO analysis of a target gene oar-let-7b of a target miRNA of the present invention; (uterus development, rhythmic process, regulatory T cell differentiation, regulation of protein phosphorylation, regulation of circadian rhythm, receptor internalization, protein kinase B signaling, positive regulation of transcription from RNA polymerase II promoter, positive regulation of phosphatidylinositol 3-kinase signaling, positive regulation of cell proliferation, positive regulation of ERK1 and ERK2 cascade, positive regulation of cell proliferation, phospholipase C-activating G-protein coupled receptor signaling pathway, negative regulation of myoblast differentiation, negative regulation of gene expression, inflammatory response, immune response, germ cell migration, epithelial cell differential, cellular response to virus, cellular response to transforming growth factor beta stimulus, basement membrane organization, adenylate cyclase-activating G-protein coupled receptor signaling pathway, adaptive immune response, plasma membrane, integral component of plasma membrane, integral component of plasma membrane, extracellular space, external side of plasma membrane, endosome membrane, cell surface, protein serine/threonine/tyrosine kinase activity, growth factor activity, chemokine activity);

FIG. 2 is a statistical graph of KEGG analysis of a target gene oar-let-7b of a target miRNA of the present invention; (cytokine-cytokine receptor interaction, pathways in cancer, PI3K-Akt signaling pathway, intestinal immune network for IgA production, Malaria, influenza A, Proteoglycans in cancer, Hypertrophic cardiomyopathy, IL-17 signalling pathway, Rheumatold arthritis, AGE-RAGE signalling pathway in diabetic complications, Hematopoletic cell lineage, Amoebiasis, Fluld shear stress and atherosclerosis, Alcoholic liver disease, Gastric cancer, Cell adhesion molecules, mTOR signalling pathway, cellular senescence, Measles, Neuroactive ligand-receptor interaction, Tight junction, NOD-like receptor signaling pathway, hepatitis B, Non-alcoholic fatty liver disease);

FIG. 3 is a statistical graph of GO analysis of a target gene oar-miR-19b of a target miRNA of the present invention; (T cell differentiation in thymus, regulation of synapse organization, positive regulation of osteoblast differentiation, positive regulation of peptidyl-serine phosphorylation, positive regulation of osteoblast differentiation, positive regulation of osteoblast differentiation, positive regulation of neuron apoptotic process, positive regulation of neuron apoptotic process, positive regulation of angiogenesis, osteoclast differentiation, negative regulation of oxidative stress-induced neuron death, negative regulation of osteoblast differentiation, negative regulation of cysteine-type endopeptidase activity involved in apoptotic process, mitochondrial transport, glutathione metabolic process, embryonic digit morphogenesis, cellular response to ionizing radiation, cellular response to amino acid stimulus, branching involved in blood vessel morphogenesis, endosome membrane, cell surface, apical part of cell, protein phosphatase binding, identical protein binding, copper ion binding);

FIG. 4 is a statistical graph of KEGG analysis of an oar-miR-19b of the present invention; (Human cytomegalovirus infection, Proteoglycans in cancer, Epstein-Barrier virus infection);

FIG. 5 is a statistical graph of GO analysis of an oar-miR-29a of the present invention; (ventricular septum morphogenesis, SMAD protein signal trasduction, regulation of mitotic nuclear division, regulation of cytokine production, positive regulation of small GTPase mediated signal transduction, positive regulation of Notch signaling pathway, positive regulation of Notch signaling pathway, positive regulation of MAPK cascade, positive regulation of MAPK cascade, positive regulation of endothelial cell proliferation, positive regulation of endothelial cell proliferation, positive regulation of endothelial cell migration, positive regulation of BMP signaling pathway, positive chemotaxis, phagocytosis, engulfment, outflow tract septum morphogenesis, negative regulation of endothelial cell proliferation, MyD88-dependent toll-like receptor signaling pathway, innate immune response, inflammatory response, ERK1 and ERK2 cascade, detection of triacyl bacterial lipopeptide, cellular response to interferon-gamma, cell adhesion, branching morphogenesis of an epithelial tube, blood vessel remodeling, transcription factor complex, transcription factor complex, Toll-like receptor 1-Toll-like receptor 2 protein complex, nucleus, integral component of plasma membrane, Golgi apparatus, extracellular space, extracellular region, external side of plasma membrane, cytoplasmic ribonucleoprotein granule, cell surface, acrosomal membrane, R-SMAD binding, protein kinase binding, protein kinase binding, protein homodimerization activity, NAD+nucleosidase activity, metal ion binding, identical protein binding, chaperone binding);

FIG. 6 is a statistical graph of KEGG analysis of an oar-miR-29a of the present invention; (Measles, Toll-like receptor signaling pathway, leishmaniasis, Adherens junction, Focal adhesion, Tuberculosis, Proteoglycans in cancer, Osteoclast differentiation, Pathways in cancer, Fluid shear and atherosclerosis, Phagosome, Thyroid hormone synthesis, Hepatitis B, Pancreatic cancer, Bacterial invasion of epithelial cells, Human papillomavirus infection, PD-L1 expression and PD-1 checkpoint pathway in cancer, Fc gamma R-mediated phagocytosis, Rheumatoid arthritis, PI3K-Akt signaling pathway, Salmonella infection, Proximal tubule blacarbonate reclamation, MAPK signaling pathway);

FIG. 7 is a PPI network interaction diagram of a predicted target gene of oar-miR-29a of the present invention;

FIG. 8 is a relative expression of oar-miR-29a after transfection of mimics of the present invention;

FIG. 9 is a relative expression of oar-miR-29a after transfection of inhibitor of the present invention;

FIG. 10 is a relative expression of CDC42 after transfection of mimics and inhibitor of the present invention;

FIG. 11 is a binding site of oar-miR-29a and the 3′ UTR of CDC42 of the present invention, where query sequence is: 3′-ttggctaaaGTCTACCACGAt-5′ shown in SEQ ID NO: 13; and a ref sequence is: 5′-catgttcccCACCTGGTGCTc-3′ shown in SEQ ID NO: 14;

FIG. 12 is a result of a dual-luciferase reporter gene assay of the present invention.

### DETAILED DESCRIPTION OF THE EMBODIMENTS

The following is a detailed introduction to the present invention in combination with drawings and specific embodiments.

A method for screening the aforementioned molecular marker, the method includes the following steps:
step 1, the differential expression of miRNA in the plasma of pregnant sheep and non-pregnant sheep is analyzed by the bioinformatics method
a composition of miRNA in plasma of pregnant sheep and non-pregnant sheep in the public database is compared, the results show that the composition of miRNA in plasma of pregnant sheep is specific, and a group of miRNA with high differential expression abundance in the pregnancy stage are screened, wherein the group of miRNA is oar-let-7b, oar-miR-19b and oar-miR-29a, respectively;

TABLE 1
Relative expression levels of miRNA in plasma
of pregnant and non-pregnant sheep

| miRNA ID | Regulation | Relative expression level |
|---|---|---|
| oar-let-7b | Downregulated | −1.78 |
| oar-miR-19b | Upregulated | 2.79 |
| oar-miR-29a | Upregulated | 1.79 |

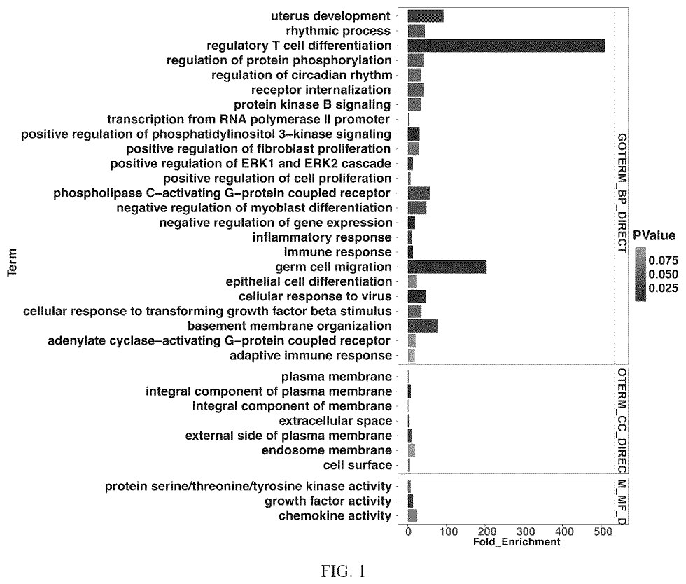

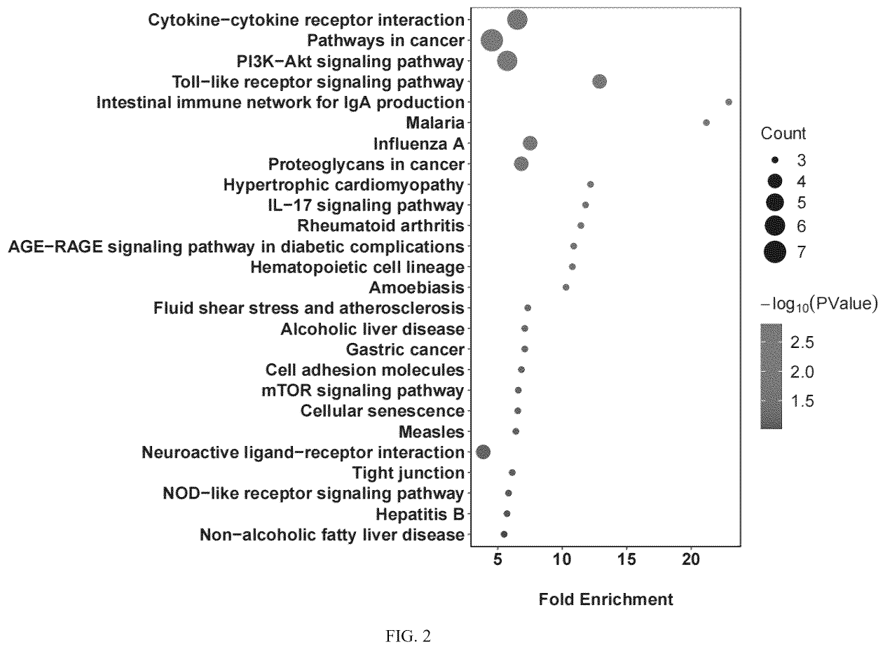

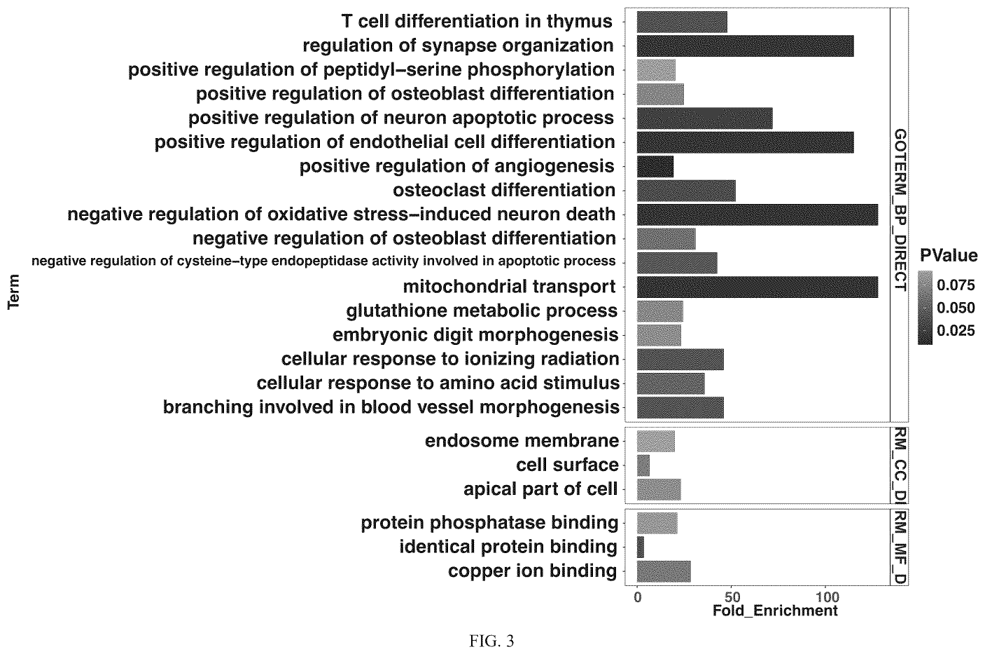

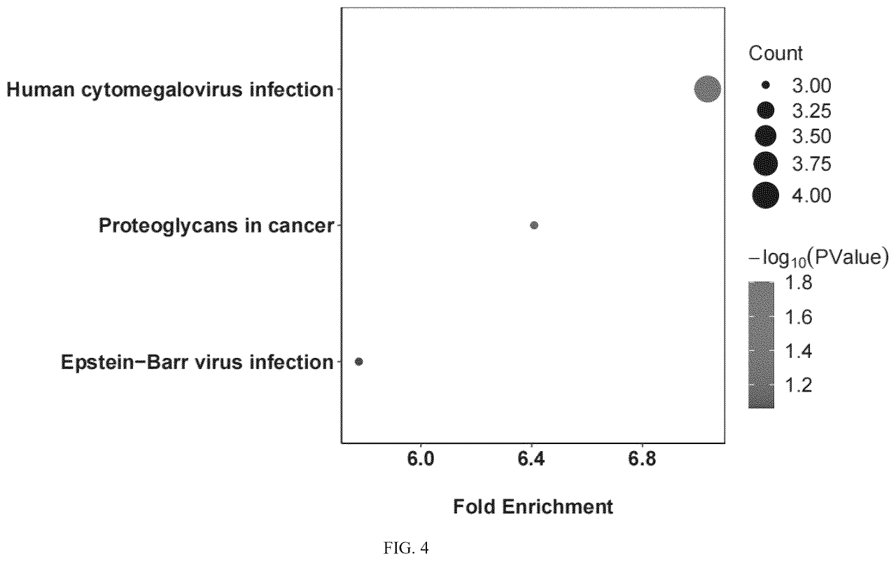

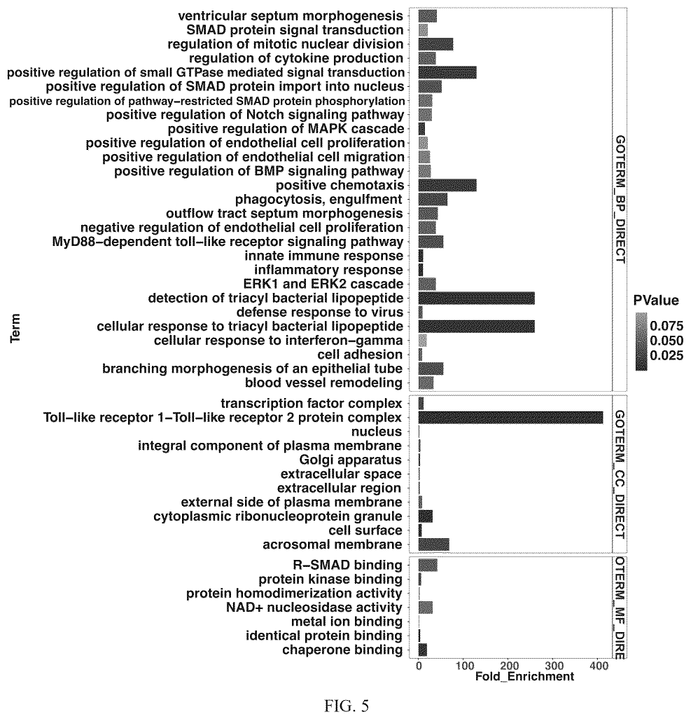

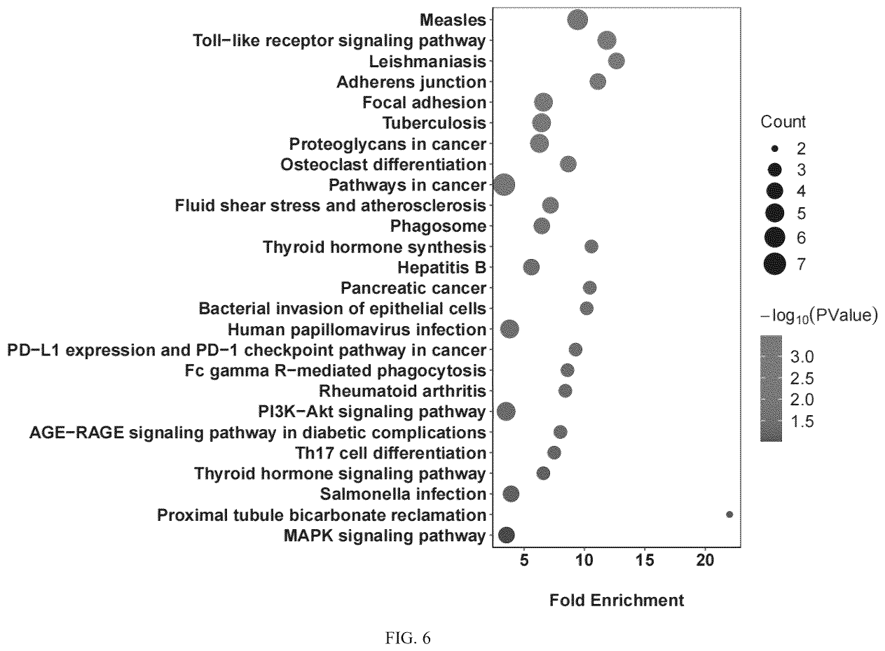

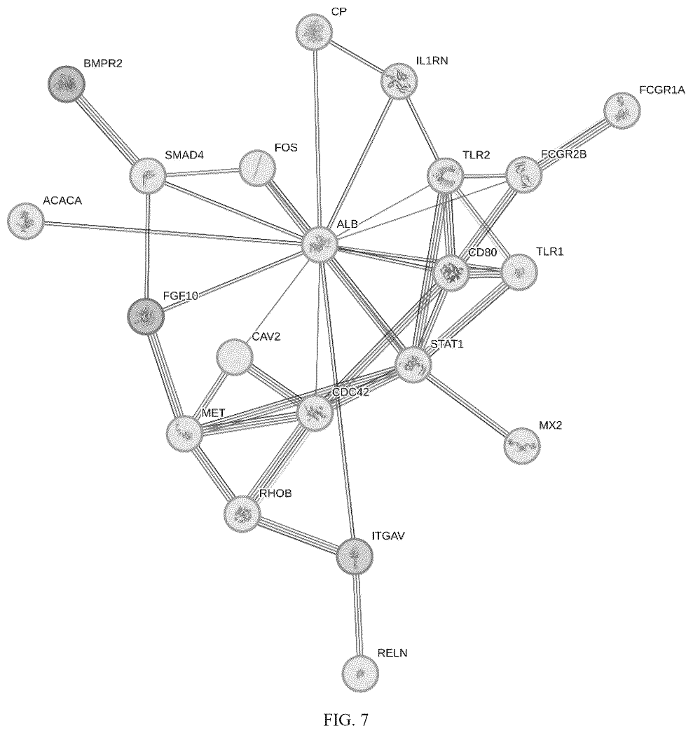

the sequence of oar-miR-29a is: 5′ UAGCACCAUUUGAAAUCAGUGUU 3′, shown in SEQ ID NO: 12.
Step 2, miRNA related to the pregnancy stage of sheep and its target genes are screened out
an online tool (bioinfo5.ugr.es/srnatoolbox) is used to predict the target gene of a target miRNA, and KEGG analysis and GO analysis (FIGS. 1-6) are performed on the target gene of the target miRNA to predict its correlation with embryo implantation, it can be seen that oar-miR-29a has a higher correlation with embryo implantation, a PPI network (FIG. 7) is constructed for its target gene, and the following four genes located at key nodes are obtained (ALB, CD80, CDC42, and STAT). Wherein, CDC42 has a higher correlation with embryo implantation according to the literature, and CDC42 is selected as the final target gene in the present invention.
Step 3, miRNA and its target genes are verified by experiments
in order to verify the accuracy of bioinformatics screening results, the final target gene CDC42 is selected, and the final target gene CDC42 is verified by fluorescence quantitative PCR in Hu sheep endometrial epithelial cells transfected with oar-miR-29a mimics and inhibitor.

TABLE 2
Cell transfection system

| Component | Volume/μL |
|---|---|
| Opti-MEM | 125 |
| RNA oligo | 7.5 |
| Lipofectamine 3000 | 7.5 |
| Opti-MEM | 125 |
| Total | 265 |

TABLE 3
Primer sequences

| Primer | Sequence (5′ to 3′) a | SEQ ID NO: |
|---|---|---|
| oar-miR-29a specific primer | CTAGCACCATCTGAAATCGGTTA | 1 |
| U6 forward primer | GGAACGATACAGAGAAGATTAGC | 2 |
| U6 reverse primer | TGGAACGCTTCACGAATTTGCG | 3 |
| CDC42 forward primer | GACCGCTGAGCTATCCAC | 4 |
| CDC42 reverse primer | CTTGACAGCCTTCAGGTCAC | 5 |

TABLE 4
qPCR reaction procedure

| Cycle | | | Cycle |
|---|---|---|---|
| step | Temperature | Time | number |
| Pre-denaturation | 95° C. | 10 min | 1 |
| Degeneration | 95° C. | 15 sec | |
| Annealing | 58° C. | 20 sec | 40 |
| Extension | 72° C. | 20 sec | |

| | | |
|---|---|---|
| Melting curve analysis | Instrument default settings | 1 |

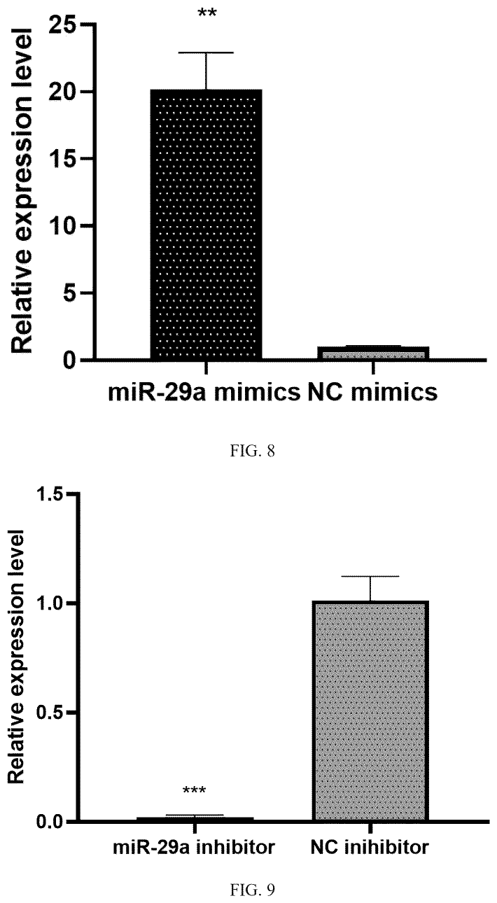

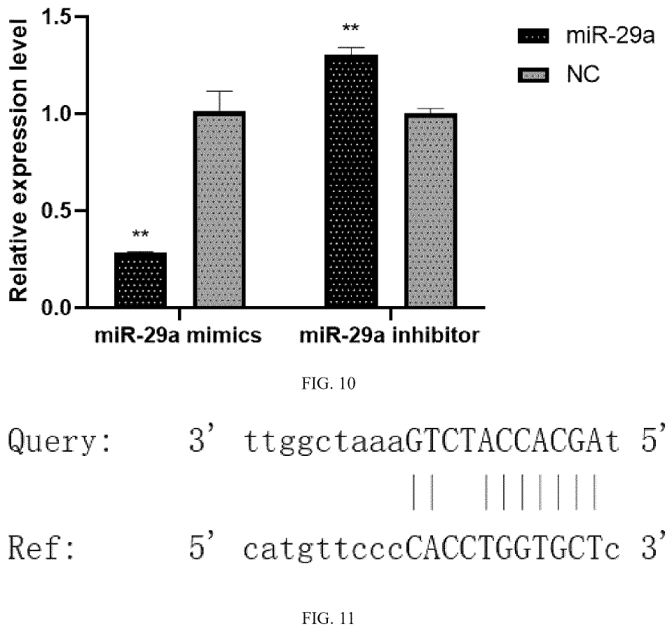

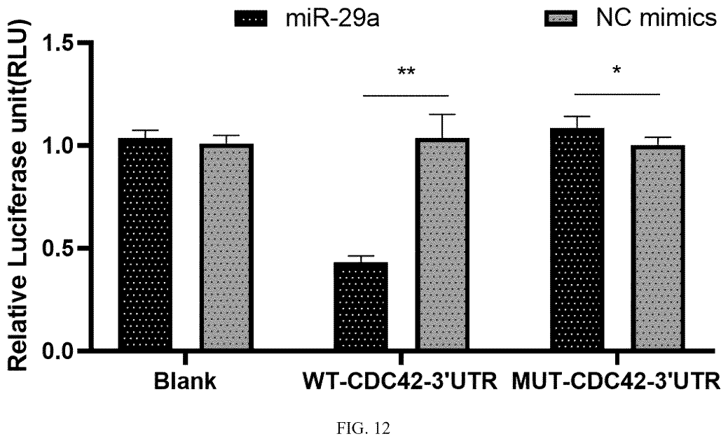

The results showed that the relative expression of oar-miR-29a was significantly increased and significantly decreased after transfection of oar-miR-29a mimics and inhibitor into Hu sheep endometrial epithelial cells (FIG. 8, and FIG. 9), while the relative expression of CDC42 was trended in opposite to the relative expression of oar-miR-29a (FIG. 10), which proved that the transfection system could effectively transfect RNAoligo into Hu sheep endometrial epithelial cells, and oar-miR-29a mimics could significantly inhibit the expression of CDC42.

A method for verifying a targeting relationship between the oar-miR-29a and CDC42, the method includes the following steps:
step 1, a binding sequence of oar-miR-29a and 3′ UTR of CDC42 is obtained from a database, the CDC42 wild type plasmid vector is constructed, that is, CDC42 WT-oar-miR-29a, and the CDC42 mutant type plasmid vector is constructed, that is, CDC42 MUT-oar-miR-29a. RNA oligo and vector were synthesized by Shanghai GenePharma Co., Ltd. The specific sequence information is shown in Table 5.

TABLE 5
RNA oligo and plasmid vector sequences

| Name | Sequence (5′ to 3′) | SEQ ID NO: |
|---|---|---|
| oar-miR-29a | UAGCACCAUCUGAAAUCGGUUCCGAUUUCAGAUGGUGCUAUU | 6 |
| mimics | | |
| oar-miR-29a | AACCGAUUUCAGAUGGUGCUA | 7 |
| inhibitor | | |
| mimics NC | UUCUCCGAACGUGUCACGUTTACGUGACACGUUCGGAGAATT | 8 |
| inhibitor NC | CAGUACUUUUGUGUAGUACAA | 9 |
| CDC42 WT-oar- | CCTAGATCTAGTTTAGAGAACATGTTCCCCACCTG | 10 |
| miR-29a | GTGCTCTTAGGAAGGAGTATAGTAAACGCCTCAT | |
| CDC42 MUT-oar- | CCTAGATCTAGTTTAGAGAACATGTTCCCGTCCAC | 11 |
| miR-29a | CACGACTTAGGAAGGAGTATAGTAAACGCCTCAT | |

oar-miR-29a mimics and mimics NC are co-transfected respectively with CDC42 wild type/mutant plasmids into HEK293T cells, the co-transfection system is shown in Table 6. After 48 hours of transfection, the fluorescence activity was identified by a dual-luciferase reporter gene detection system.

TABLE 6
RNA oligo, plasmid vector co-transfection system

| Component | Volume/μL |
|---|---|
| Opti-MEM | 25 |
| RNA oligo | 0.75 |
| P3000 | 2 |
| Plasmid vector | 1 |
| Lipofectamine 3000 | 1.5 |
| Opti-MEM | 25 |
| Total | 55.25 |

The results are shown in FIG. 12, the fluorescence activity of CDC42 wild type plasmid and oar-miR-29a mimics co-transfection group was significantly reduced, which proved that oar-miR-29a targeted CDC42.

In summary, the present invention provides a reliable method for predicting and verifying differentially expressed miRNAs and their target genes in sheep during pregnancy. Through integrated bioinformatics analysis and experimental verification, the miRNA and its target gene related to sheep pregnancy can be accurately identified, which provides important clues for further understanding of sheep pregnancy regulatory network, and provides new methods and approaches for improving sheep breeding efficiency.

The above shows and describes the basic principles, main features and advantages of the present invention. Technicians in the technical field should understand that the above-mentioned embodiments do not limit the present invention in any way, and the technical solutions obtained by equivalent substitution or equivalent transformation should fall within the scope of protection of the present invention.

What is claimed is:

1. A molecular marker related to a differential expression of a pregnancy stage of sheep, wherein the molecular marker is an oar-miR-29a miRNA target gene, and a sequence of the oar-miR-29a miRNA target gene is as follows: 5′-UAGCACCAUUUGAAAUCAGUGUU-3′ shown in SEQ ID NO: 12.

2. The molecular marker related to the differential expression of the pregnancy stage of the sheep according to claim 1, wherein the oar-miR-29a miRNA target gene targets cell division cycle 42 (CDC42), and oar-miR-29a mimics inhibit a CDC42 expression.

3. A method for screening the molecular marker according to claim 1, comprising the following steps:
step 1, analyzing a differential expression of miRNA in a plasma of pregnant sheep and miRNA in a plasma of non-pregnant sheep by a bioinformatics method:
comparing a composition of the miRNA in the plasma of the pregnant sheep and a composition of the miRNA in the plasma of the non-pregnant sheep in a public database to obtain results, wherein the results show that the composition of the miRNA in the plasma of the pregnant sheep is specific, and a group of miRNA with a high differential expression abundance in the pregnancy stage are screened, wherein the group of the miRNA with the high differential expression abundance in the pregnancy stage is oar-let-7b, oar-miR-19b, and oar-miR-29a, respectively;
step 2, screening out miRNA related to the pregnancy stage of the sheep and target genes of the miRNA related to the pregnancy stage of the sheep:
using an online tool to predict a target gene of a target miRNA, and performing a Kyoto Encyclopedia of Genes and Genomes (KEGG) analysis and a Gene Ontology (GO) analysis on the target gene of the target miRNA to predict a correlation between the target gene of the target miRNA and an embryo implantation, wherein the oar-miR-29a has a higher correlation with the embryo implantation than either the oar-let-7b or the oar-miR-19b, constructing a protein-protein interaction (PPI) network for a target gene of the oar-miR-29a, and obtaining the following four genes located at key nodes: Albumin (ALB), Cluster of differentiation (CD) 80, CDC42, and signal transducer and activator of transcription (STAT); and selecting the CDC42 with a higher correlation with the embryo implantation than the ALB, the CD, or the STAT as a final target gene;
step 3, verifying the miRNA related to the pregnancy stage of the sheep and the target genes of the miRNA related to the pregnancy stage of the sheep by experiments:
verifying the target gene CDC42 by a fluorescence quantitative polymerase chain reaction (PCR) in Hu sheep endometrial epithelial cells transfected with oar-miR-29a mimics and an oar-miR-29a inhibitor; wherein PCR primer sequences comprise: oar-miR-29a specific primer: CTAGCACCATCTGAAATCGGTTA shown in SEQ ID NO: 1; U6 forward primer GGAACGATACAGAGAAGATTAGC shown in SEQ ID NO: 2; U6 reverse primer TGGAACGCTTCACGAATTTGCG shown in SEQ ID NO: 3; CDC42 forward primer GACCGCTGAGCTATCCAC shown in SEQ ID NO: 4; and CDC42 reverse primer CTTGACAGCCTTCAGGTCAC shown in SEQ ID NO: 5;
wherein after the Hu sheep endometrial epithelial cells are transfected with the oar-miR-29a mimics and the oar-miR-29a inhibitor, a relative expression of the oar-miR-29a increases and decreases, respectively, while a relative expression of the CDC42 is trended in opposite to the relative expression of the oar-miR-29a.

4. A method for verifying a targeting relationship between the molecular marker according to claim 1 and CDC42, comprising the following steps:
step 1, obtaining a binding sequence of oar-miR-29a and 3′ UTR of the CDC42 from a database, constructing a CDC42 wild type (WT) plasmid vector, that is, CDC42 WT-oar-miR-29a, and constructing a CDC42 mutant type (MUT) plasmid vector, that is, CDC42MUT-oar-miR-29a;
oar-miR-29a mimics sequence:
UAGCACCAUCUGAAAUCGGUUCCGAUUUCAGAUGGUGCUAUU shown in SEQ ID NO: 6;
oar-miR-29a inhibitor sequence: AACCGAUUUCAGAUGGUGCUA shown in SEQ ID NO: 7;
mimics negative control (NC) sequence: UUCUCCGAACGUGUCACGUTT ACGUGACACGUUCGGAGAATT shown in SEQ ID NO: 8;
inhibitor NC sequence: CAGUACUUUUGUGUAGUACAA shown in SEQ ID NO: 9;
CDC42 WT-oar-miR-29a sequence:
CCTAGATCTAGTTTAGAGAACATGTTCCCCACCTGGTGCTCTTAGGAAGGAG TATAGTA AACGCCTCAT shown in SEQ ID NO: 10;
CDC42 MUT-oar-miR-29a sequence:
CCTAGATCTAGTTTAGAGAACATGTTCCCGTCCACCACGACTTAGGAAGGAG TATAGT AAACGCCTCAT shown in SEQ ID NO: 11;
step 2, co-transfecting oar-miR-29a mimics and a mimics NC respectively with a CDC42 wild type plasmid and a CDC42 mutant plasmid into human embryonic kidney (HEK293T) cells to obtain the transfected HEK293T cells, and detecting a fluorescence activity of the transfected HEK293T cells by a dual-luciferase reporter gene assay system after a transfection; and
wherein the fluorescence activity of a co-transfection group of the CDC42 wild type plasmid and the oar-miR-29a mimics is decreased.


SEQUENCE LISTING (14 sequences)

SEQ ID NO: 1 - DNA, 23 bp, synthetic construct
  source 1..23: mol_type = other DNA
ctagcaccatctgaaatcggtta

SEQ ID NO: 2 - DNA, 23 bp, synthetic construct
  source 1..23: mol_type = other DNA
ggaacgatacagagaagattagc

SEQ ID NO: 3 - DNA, 22 bp, synthetic construct
  source 1..22: mol_type = other DNA
tggaacgcttcacgaatttgcg

SEQ ID NO: 4 - DNA, 18 bp, synthetic construct
  source 1..18: mol_type = other DNA
gaccgctgagctatccac

SEQ ID NO: 5 - DNA, 20 bp, synthetic construct
  source 1..20: mol_type = other DNA
cttgacagccttcaggtcac

SEQ ID NO: 6 - RNA, 42 nt, synthetic construct
  source 1..42: mol_type = other RNA
tagcaccatctgaaatcggttccgatttcagatggtgctatt

SEQ ID NO: 7 - RNA, 21 nt, synthetic construct
  source 1..21: mol_type = other RNA
aaccgatttcagatggtgcta

SEQ ID NO: 8 - RNA, 42 nt, synthetic construct
  source 1..42: mol_type = other RNA
ttctccgaacgtgtcacgtttacgtgacacgttcggagaatt

SEQ ID NO: 9 - RNA, 21 nt, synthetic construct
  source 1..21: mol_type = other RNA
cagtacttttgtgtagtacaa

SEQ ID NO: 10 - DNA, 69 bp, synthetic construct
  source 1..69: mol_type = other DNA
cctagatctagtttagagaacatgttccccacctggtgctcttaggaaggagtatagtaaacgcctcat

SEQ ID NO: 11 - DNA, 69 bp, synthetic construct
  source 1..69: mol_type = other DNA
cctagatctagtttagagaacatgttcccgtccaccacgacttaggaaggagtatagtaaacgcctcat

SEQ ID NO: 12 - RNA, 23 nt, synthetic construct
  source 1..23: mol_type = other RNA
tagcaccatttgaaatcagtgtt

SEQ ID NO: 13 - DNA, 21 bp, synthetic construct
  source 1..21: mol_type = other DNA
ttggctaaagtctaccacgat

SEQ ID NO: 14 - DNA, 21 bp, synthetic construct
  source 1..21: mol_type = other DNA
catgttccccacctggtgctc